(19) United States
(12) Patent Application Publication
(10) Pub. No.: US 2026/0251590 A1
(43) Pub. Date: Aug. 27, 2026

(54) METHOD, ELECTRONIC DEVICE, AND SYSTEM FOR DETECTING DEFECT OF SOLDER PASTE

(71) Applicant: Wiwynn Corporation, New Taipei City (TW)

(72) Inventors: Jia Ying Zhang, New Taipei City (TW); Gewei Fang, New Taipei City (TW); Jui-Che Hsu, New Taipei City (TW); Yu-Fong Su, New Taipei City (TW); Hsuehwen Chang, New Taipei City (TW); Shih-Yi Chao, New Taipei City (TW)

(73) Assignee: Wiwynn Corporation, New Taipei City (TW)

(21) Appl. No.: 19/224,920

(22) Filed: Jun. 2, 2025

(30) Foreign Application Priority Data
Feb. 27, 2025 (TW) 114107464

(51) Int. Cl. G01N 23/18; G01N 23/04; G01N 23/083; G01N 33/207; G06T 7/00; G06V 10/25; G06V 10/774; G06V 10/776; G06V 10/82; G06V 20/70

(52) U.S. Cl. CPC G01N 23/18; G01N 23/04; G01N 23/083; G01N 33/207; G06T 7/0004; G06V 10/25; G06V 10/774; G06V 10/776; G06V 10/82; G06V 20/70; G01N 2223/401; G01N 2223/629; G01N 2223/646; G06T 2207/10116; G06T 2207/20081; G06T 2207/20084; G06T 2207/30141; G06T 2207/30152

(57) ABSTRACT

A method, an electronic device, and a system for detecting a defect of a solder paste are provided. The method includes: receiving a first image and a label from an automated X-ray inspection machine, wherein the label indicates a bounding box of the solder paste; enlarging the bounding box to update the bounding box; generating a detection result of the defect of the solder paste according to the first image and the bounding box using a machine learning model; and outputting the detection result.

### CROSS-REFERENCE TO RELATED APPLICATION

This application claims the priority benefit of Taiwan application serial no. 114107464, filed on Feb. 27, 2025. The entirety of the above-mentioned patent application is hereby incorporated by reference herein and made a part of this specification.

### BACKGROUND

### Technical Field

The disclosure relates to an image recognition technology, and more particularly to a method, an electronic device, and a system for detecting a defect of a solder paste.

### Description of Related Art

The printed circuit board includes various electronic elements such as capacitors, resistors, transistors, chips, connectors, or dual in-line package (DIP) elements that are soldered using surface mount technology (SMT). The solder paste for soldering may have defects such as foreign matter or incomplete filling. Since the defects are difficult to be recognized with the naked eye, an automated X-ray inspection (AXI) machine is used for recognizing the defects. However, in actual applications, the AXI machine often judges a solder paste in good condition as defective, causing an excessively high overkill rate. As a result, factories waste unnecessary manpower to conduct reinspection of defects of solder pastes.

### SUMMARY

The disclosure provides a method, an electronic device, and a system for detecting a defect of a solder paste, which can reduce an overkill rate of defect recognition of the solder paste.

The disclosure provides a method for detecting a defect of a solder paste including steps of receiving a first image and a label of the solder paste are received from an automated X-ray inspection machine, in which the label indicates a bounding box of the solder paste; enlarging the bounding box to update the bounding box; by a machine learning model, generating a detection result of the defect of the solder paste according to the first image and the bounding box; and outputting the detection result.

The disclosure further provides an electronic device for detecting a defect of a solder paste and including a communication interface, a processor, and a memory. The communication interface is configured to receive at least one image and a label of the solder paste. The processor is electrically connected to the communication interface. The memory is electrically connected to the processor and is configured to store a program code. The program code instructs the processor to execute the method for detecting the defect of the solder paste according to the at least one image and the label.

The disclosure further provides a system for detecting a defect of a solder paste including an automated X-ray inspection machine, the electronic device as abovementioned, an electronic inspection station, and a manual inspection station. The automated X-ray inspection machine is configured to capture at least one image of the solder paste of a circuit board and generate a label of the at least one image. The electronic device is connected to the automated X-ray inspection machine, and is configured to generate a detection result of the defect of the solder paste according to the at least one image and the label. The electronic inspection station is connected to the electronic device and is configured to receive the circuit board when the detection result indicates “qualified”. The manual inspection station is connected to the electronic device and is configured to receive the circuit board when the detection result indicates “unqualified”.

The method, the electronic device, and the system for detecting the defect of the solder paste of the disclosure have the following characteristics. (1) The AXI machine and the electronic device are configured to double check defect inspection. If the detection result of the second defect inspection is still unqualified, personnel at the manual inspection station will perform the third inspection to prevent unnecessary manual inspection. (2) Multiple machine learning models are configured to respectively perform defect inspection on multiple solder pastes of multiple solder paste groups to solve the issue of the overkill rate of a single machine learning model being too high. (3) Pre-processing such as enlarging the bounding box of the input image may be performed, which may solve the issue of the bounding box generated by the conventional AXI machine being displaced or inaccurate. (4) For a thicker solder paste, such as a ball grid array or a plated through hole, multiple images corresponding to multiple cross-sections are concatenated for image recognition, which may fully inspect the defect of the solder paste. (5) For the thicker solder paste, weights of neurons in two Siamese networks used are exactly the same, which may save memory spaces occupied by the neural networks.

### BRIEF DESCRIPTION OF THE DRAWINGS

FIG. 1 is a schematic diagram of a system for detecting a defect of a solder paste according to an embodiment of the disclosure.

FIG. 2A is a schematic diagram of a table of qualified samples and unqualified samples of a first solder paste group according to an embodiment of the disclosure.

FIG. 2B is a schematic diagram of a table of qualified samples and unqualified samples of a second solder paste group according to an embodiment of the disclosure.

FIG. 2C is a schematic diagram of a table of qualified samples and unqualified samples of a third solder paste group according to an embodiment of the disclosure.

FIG. 3 is a flowchart of a method for detecting a defect of a solder paste according to an embodiment of the disclosure.

FIG. 4 is a schematic diagram of image recognition of the first solder paste group according to an embodiment of the disclosure.

FIG. 5 and FIG. 6 are respectively a schematic diagram and a flowchart of a training process of a third machine learning model according to an embodiment of the disclosure.

FIG. 7 and FIG. 8 are respectively a schematic diagram and a flowchart of an inference process of the third machine learning model according to an embodiment of the disclosure.

FIG. 9 is a schematic diagram of an electronic device according to an embodiment of the disclosure.

FIG. 10 is a flowchart of a method for detecting a defect of a solder paste according to an embodiment of the disclosure.

### DESCRIPTION OF THE EMBODIMENTS

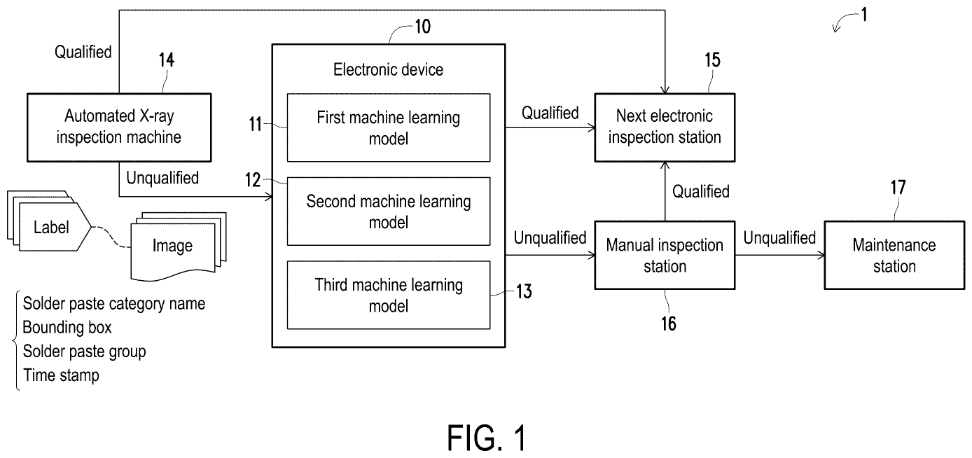

FIG. 1 is a schematic diagram of a system 1 for detecting a defect of a solder paste according to an embodiment of the disclosure. The system 1 may include an automated X-ray inspection (hereinafter abbreviated AXI) machine 14, an electronic device 10, a next electronic inspection station 15, a manual inspection station 16, and a maintenance station 17. The AXI machine 14 is configured to capture at least one image of a solder paste of a circuit board and generate a label of the at least one image. The electronic device 10 is connected to the AXI machine 14 and is configured to generate a detection result of a defect of the solder paste according to the at least one image and the label. The next electronic inspection station 15 is connected to the electronic device 10 and is configured to receive the circuit board when the detection result indicates that the circuit board is qualified. The manual inspection station 16 is connected to the electronic device 10 and the next electronic inspection station 15, and is configured to receive the circuit board when the detection result indicates that the circuit board is unqualified, so that personnel may manually inspect the circuit board. The maintenance station 17 is connected to the manual inspection station 16, and is configured to receive a printed circuit board from the manual inspection station 16, so that personnel may maintain the printed circuit board. In an embodiment, the system 1 may include a conveyor (not shown) configured to transport the circuit board to a designated machine and inspection station.

In practical applications, the AXI machine 14 may perform photography, image recognition, and first defect inspection on the solder paste on the printed circuit board. If the solder paste has no defect, the AXI machine 14 records “qualified” on the label, and the conveyor automatically transports the printed circuit board to the next electronic inspection station 15. If the solder paste has a defect, the AXI machine 14 records “unqualified” and a bounding box corresponding to the defect on the label, and then transmits the image of the solder paste and the corresponding label to the electronic device 10. It should be understood that because the overkill rate of the conventional AXI machine 14 is too high, the disclosure uses the electronic device 10 to perform second defect inspection. If a detection result of the second defect inspection is still unqualified, personnel at the manual inspection station 16 will perform third inspection, which prevents unnecessary manual inspection.

Furthermore, the Applicant noted that because solder pastes come in various shapes and the criteria for judging defects are inconsistent, a single machine learning model is difficult to adapt to the diverse judgment criteria, causing a high overkill rate. Therefore, the electronic device 10 includes multiple machine learning models to respectively perform defect inspection on multiple solder pastes of multiple solder paste groups, so as to solve the issue of the overkill rate of a single machine learning model being too high.

Specifically, the AXI machine 14 may judge a solder paste category name and the corresponding solder paste group of the solder paste, and record those information in the label. If the label indicates that the solder paste belongs to a first solder paste group, the electronic device 10 uses a first machine learning model 11 to perform image recognition; if the label indicates that the solder paste belongs to a second solder paste group, the electronic device 10 uses a second machine learning model 12 to perform image recognition; and if the label indicates that the solder paste belongs to a third solder paste group, the electronic device 10 uses a third machine learning model 13 to perform image recognition.

In an embodiment, the label may indicate the solder paste category name, the solder paste group, two diagonal coordinates of the bounding box, a time stamp, and other relevant information, from which personnel can read production line data.

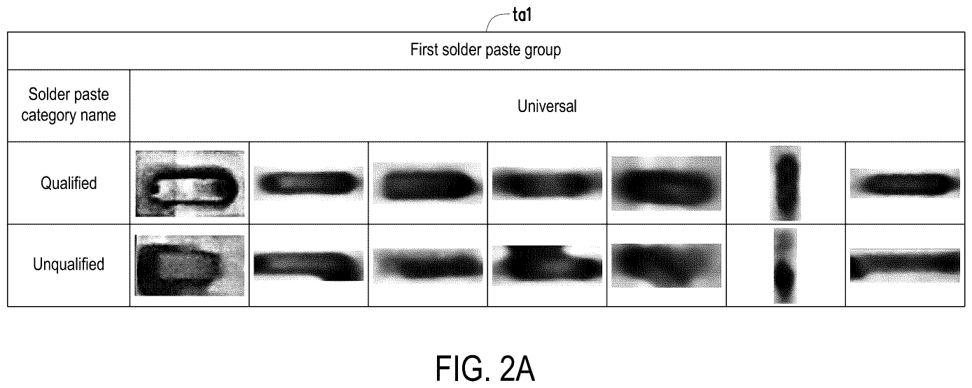

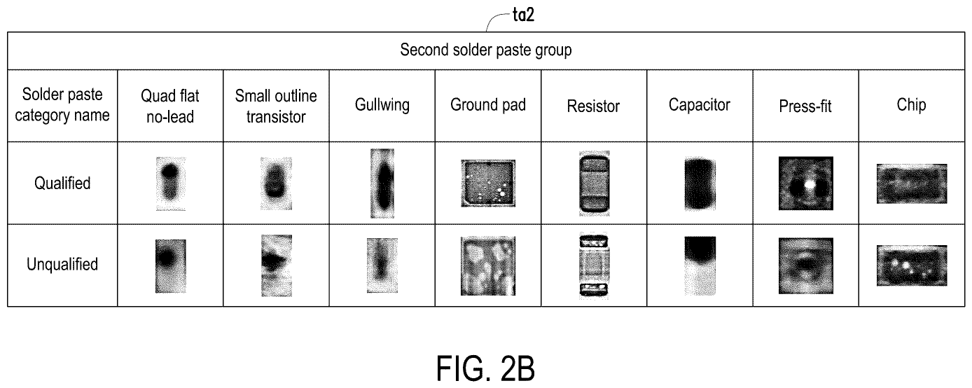

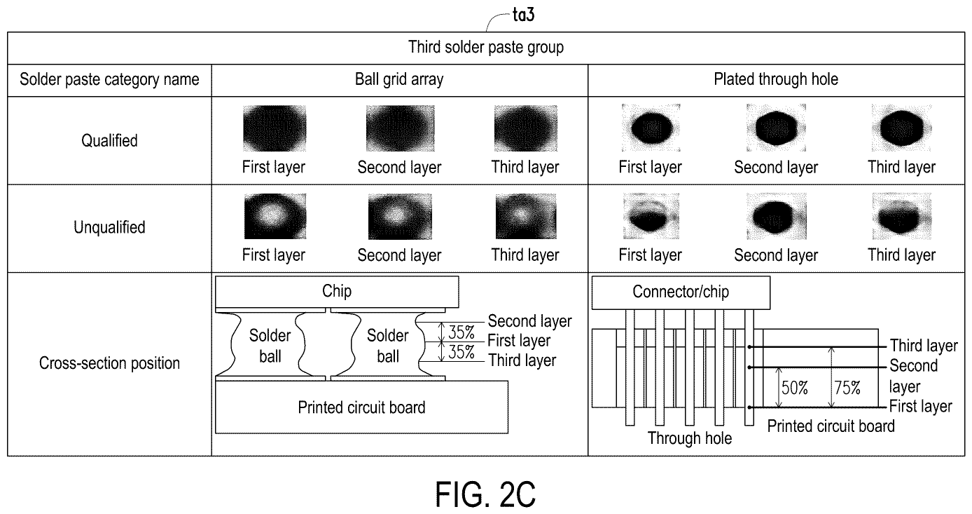

In an embodiment, one solder paste group corresponds to at least one solder paste category name. Specifically, FIG. 2A is a schematic diagram of a table ta1 of qualified samples (i.e., solder pastes without defects) and unqualified samples (i.e., solder pastes with defects) of the first solder paste group according to an embodiment of the disclosure. The solder paste category name of the solder paste belonging to the first solder paste group includes universal, which is not limited.

FIG. 2B is a schematic diagram of a table ta2 of qualified samples and unqualified samples of the second solder paste group according to an embodiment of the disclosure. The solder paste category name of the solder paste belonging to the second solder paste group includes quad flat no-lead (QFN), small outline transistor (SOT), gullwing, ground pad, resistor, capacitor, press-fit, and chip, which is not limited.

FIG. 2C is a schematic diagram of a table ta3 of qualified samples and unqualified samples of the third solder paste group according to an embodiment of the disclosure. The solder paste category name of the solder paste belonging to the third solder paste group includes ball grid array (BGA) and plated through hole (PTH), which is not limited.

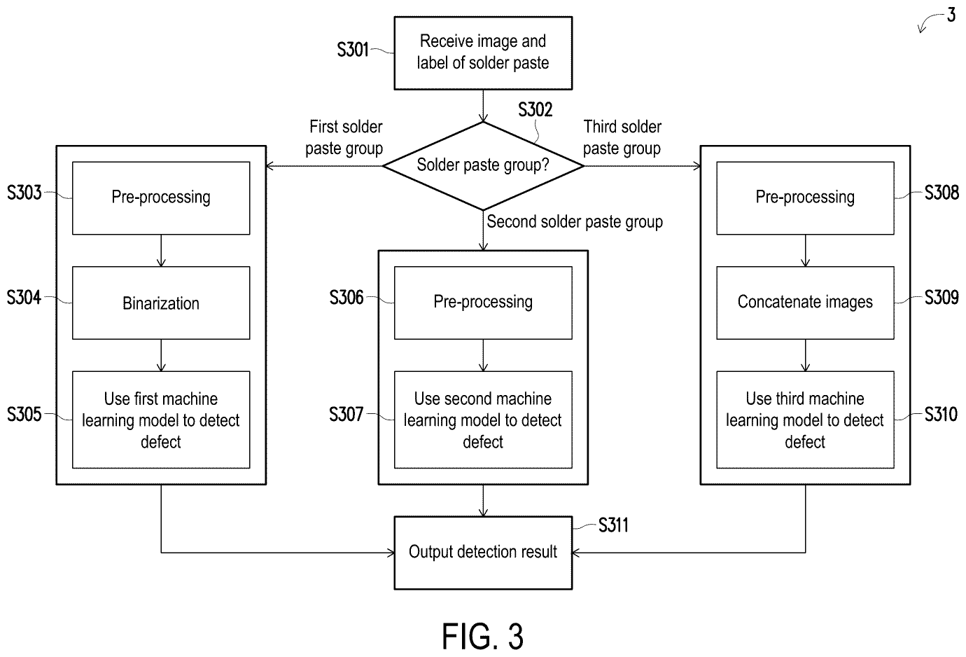

FIG. 3 is a flowchart of a method 3 for detecting a defect of a solder paste according to an embodiment of the disclosure, wherein the method 3 may be implemented by the electronic device 10.

In step S301, the electronic device 10 may receive the at least one image and the label of the solder paste from the AXI machine 14, and then read the information in the label.

In step S302, if the label indicates that the solder paste belongs to the first solder paste group, the electronic device 10 executes steps S303 to S305; if the label indicates that the solder paste belongs to the second solder paste group, the electronic device 10 executes steps S306 and S307; and if the label indicates that the solder paste belongs to the third solder paste group, the electronic device 10 executes steps S308 to S310. Finally, in step S311, the electronic device 10 may output the corresponding detection result for the at least one image and the label of the solder paste.

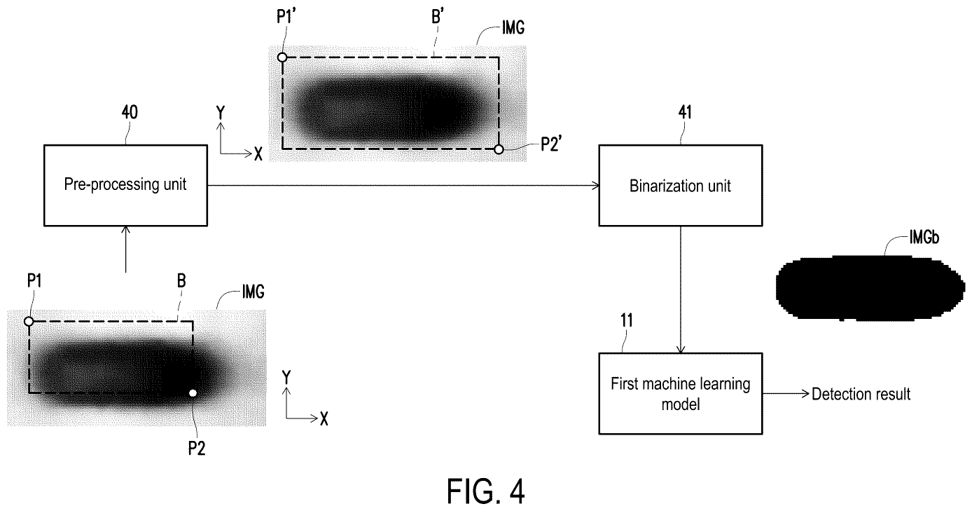

FIG. 4 is a schematic diagram of image recognition of the first solder paste group according to an embodiment of the disclosure. Please refer to FIG. 3 and FIG. 4. When the electronic device 10 receives an image IMG and the label of the solder paste from the AXI machine 14, two diagonal coordinates P1 and P2 of a bounding box B may be read, i.e., the range of the bounding box B may be defined by the upper left coordinate P1 and the lower right coordinate P2.

In step S303, the electronic device 10 pre-processes the image of the solder paste. In detail, a pre-processing unit 40 of the electronic device 10 may enlarge the bounding box B to update the bounding box B into a bounding box B′. The pre-processing unit 40 may adjust the value of at least one of the diagonal coordinates P1 and P2 to generate updated diagonal coordinates P1′ and P2′. Therefore, the pre-processing unit 40 may define the updated bounding box B′ according to the updated diagonal coordinates P1′ and P2′. It should be understood that when the AXI machine 14 performs defect inspection on the solder paste, the bounding box generated by the AXI machine 14 may be displaced or inaccurate, which may affect the accuracy of image recognition for the bounding box. In order to solve the above issue, the electronic device 10 of the disclosure may execute pre-processing including enlarging the bounding box generated by the AXI machine.

In step S304, a binarization unit 41 of the electronic device 10 may perform binarization processing on multiple pixels of the image IMG in the bounding box B′ to generate a binarized image IMGb. For example, the binarization unit 41 may perform statistical calculations on the grayscale values of the pixels according to Otsu's method to select a threshold for distinguishing the foreground from the background, and then execute binarization processing according to the threshold to generate the binarized image IMGb. In the binarized image IMGb, the pixels belonging to the foreground are set to black (the grayscale value of black is 0), and the pixels belonging to the background are set to white (the grayscale value of white is 255). Furthermore, the binarization unit 41 may set the black pixels to binary 1 and set the white pixels to binary 0 to generate the binarized image IMGb represented by binary bits.

In step S305, since the label indicates that the solder paste belongs to the first solder paste group, the electronic device 10 uses the first machine learning model 11 to perform image recognition on the binarized image IMGb to generate the detection result of the defect. The first machine learning model 11 may include a MobileNetV3 model, which is not limited. In an embodiment, the electronic device 10 may receive a historical image and a historical label of the solder paste, wherein the historical label may mark the bounding box of the defect of the solder paste on the historical image. The electronic device 10 may train the first machine learning model 11 according to the historical image and the historical label based on a supervised learning algorithm.

Please refer to FIG. 3. In step S306, the electronic device 10 may enlarge the bounding box on the image of the solder paste to update the bounding box. The method for enlarging the bounding box may be similar to the method of step S303, so there will be no reiteration.

In step S307, since the label indicates that the solder paste belongs to the second solder paste group, the electronic device 10 uses the second machine learning model 12 to perform image recognition on a part of the image in the bounding box to generate the detection result of the defect. The second machine learning model 12 may include the MobileNetV3 model, which is not limited. In an embodiment, the electronic device 10 may receive the historical image and the historical label of the solder paste, wherein the historical label may mark the bounding box of the defect of the solder paste on the historical image. The electronic device 10 may train the second machine learning model 12 according to the historical image and the historical label based on the supervised learning algorithm.

In an embodiment, the second machine learning model 12 may be configured in the AXI machine 14. The electronic device 10 may transmit the part of the image in the bounding box to the AXI machine 14. The AXI machine 14 may perform image recognition on the part according to the second machine learning model 12 or a traditional model of the AXI machine 14, thereby generating the detection result of the defect. The electronic device 10 may receive the detection result of the defect from the AXI machine 14.

Please refer to FIG. 3. In step S308, the electronic device 10 may enlarge the bounding box on the image of the solder paste to update the bounding box. The method for enlarging the bounding box may be similar to the method of step S303, so there will be no reiteration.

In step S309, the electronic device 10 may concatenate multiple images of the solder paste to generate a concatenated image. Specifically, the electronic device 10 may receive the multiple images of the same solder paste from the AXI machine 14, wherein the multiple images may correspond to the same label. The multiple images are multiple cross-sections of the same solder paste, such as a first cross-section, a second cross-section, and a third cross-section as shown in FIG. 2C, of the same solder paste.

In step S310, since the label indicates that the solder paste belongs to the third solder paste group, the electronic device 10 uses the third machine learning model 13 to perform image recognition on a part of the concatenated image in the bounding box to generate the detection result of the defect.

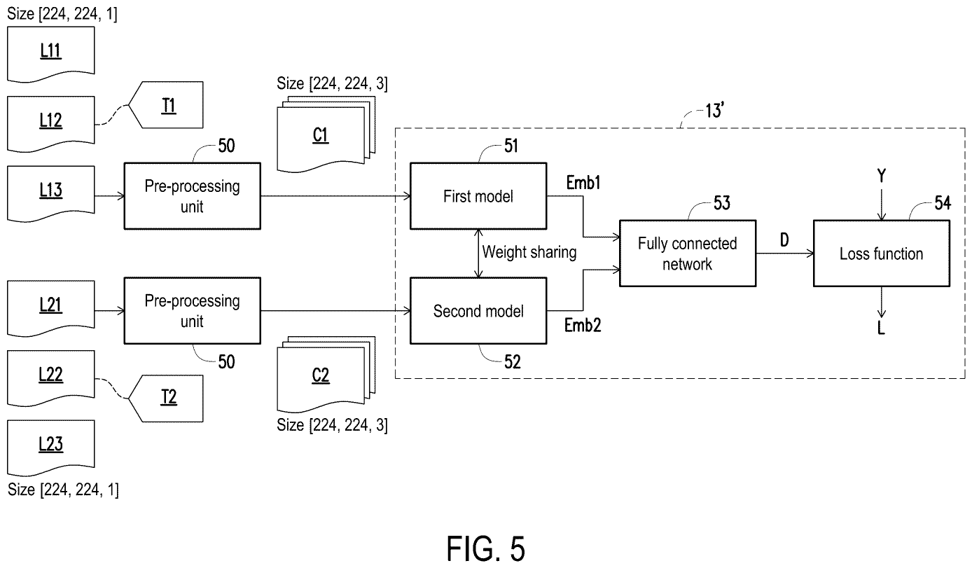

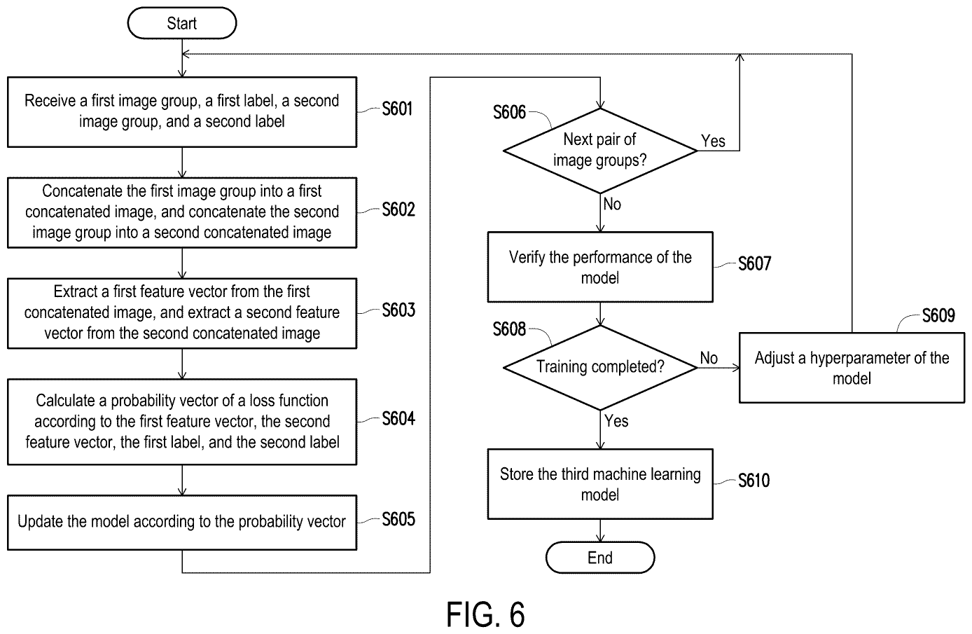

FIG. 5 and FIG. 6 are respectively a schematic diagram and a flowchart of a training process of a third machine learning model 13′ according to an embodiment of the disclosure. The third machine learning model 13′ during training includes a first model 51, a second model 52, and a fully connected network 53, wherein the first model 51 and the second model 52 are Siamese network models.

In step S601, the electronic device 10 may receive a first image group, a first label T1 corresponding to the first image group, a second image group, and a second label T2 corresponding to the second image group, wherein the first image group may include images L11, L12, and L13 corresponding to different cross-sections of one solder paste, and the second image group may include images L21, L22, and L23 corresponding to different cross-sections of another solder paste. The first label T1 may indicate bounding boxes of the images L11, L12, and L13 of the first image group, the solder paste category name, and whether the first image group has a defect. The second label T2 may indicate bounding boxes of the images L21, L22, and L23 of the second image group, the solder paste category name, and whether the second image group has a defect.

In step S602, a pre-processing unit 50 of the electronic device 10 may concatenate each image (for example, the image L11, L12, or L13) in the first image group into a first concatenated image C1, and may concatenate each image (for example, the image L21, L22, or L23) in the second image group into a second concatenated image C2.

In an embodiment, before concatenating, the pre-processing unit 50 may enlarge the bounding box on the image group or the concatenated image to update the bounding box. The method for enlarging the bounding box may be similar to the method of step S303, so there will be no reiteration.

In step S603, the first model 51 may extract a first feature vector (also known as embedding set) Emb1 from the first concatenated image C1. The second model 52 may extract a second feature vector Emb2 from the second concatenated image C2. Specifically, the electronic device 10 may input a part of the first concatenated image C1 in the bounding box into the first model 51. The first model 51 may extract the first feature vector Emb1 from the part through a neural network structure such as a convolutional layer or a pooling layer. The electronic device 10 may input a part of the second concatenated image C2 in the bounding box into the second model 52. The second model 52 may extract the second feature vector Emb2 from the part through a neural network structure such as a convolutional layer or a pooling layer.

In step S604, the electronic device 10 may calculate a probability vector L of a loss function 54 according to the first feature vector Emb1, the second feature vector Emb2, the first label T1, and the second label T2. Specifically, the fully connected network 53 of the electronic device 10 may generate a spatial vector D according to the first feature vector Emb1 and the second feature vector Emb2. In an embodiment, the sizes of the first feature vector Emb1, the second feature vector Emb2, and the spatial vector D are [64, 1]. The spatial vector D may indicate the similarity or the distance between the first feature vector Emb1 and the second feature vector Emb2. For example, the spatial vector D may be the Euclidean distance between the first feature vector Emb1 and the second feature vector Emb2. The electronic device 10 may input the spatial vector D into the loss function 54 to generate the probability vector L. In an embodiment, the size of the probability vector L is [1, 2], which respectively represent the probabilities of being qualified and unqualified.

In an embodiment, the loss function 54 may include a contrastive loss function, as shown in formula (1):

$$L = Y \cdot {D}^{2} + ( 1 - Y ) \cdot max {( m - D , 0 )}^{2} \qquad ( 1 )$$

where L is the probability vector, Y is the label value, D is the Euclidean distance between the first feature vector Emb1 and the second feature vector Emb2, and m is the boundary threshold. The boundary threshold m may be user-defined. When the first label T1 and the second label T2 are identical, the label value Y may be 1. When the first label T1 and the second label T2 are different, the label value Y may be 0. When the first label T1 indicates that the first image group has a defect and the second label T2 indicates that the second image group has a defect, the label value Y may be 1. When the first label T1 indicates that the first image group has a defect but the second label T2 indicates that the second image group does not have a defect, the label value Y may be 0.

In step S605, the electronic device 10 may update at least one of the first model 51 (or the second model 52) and the fully connected network 53 according to the probability vector L of the loss function 54. In an embodiment, the electronic device 10 may adjust weights (for example, weights of neurons in neural networks) of the first model 51 and the second model 52 based on weight sharing. In other words, the weights of the neurons of the first model 51 and the second model 52 are exactly the same, which may save memory spaces occupied by the neural networks.

In step S606, the electronic device 10 may judge whether there is a next pair of image groups in a training data set. If there is the next pair of image groups, step S601 is executed again. If there is no next pair of image groups, step S607 is executed.

In step S607, the electronic device 10 may verify the performance of the model. Specifically, the electronic device 10 may use the third machine learning model 13′ to perform image recognition on a verification data set to generate the detection result of the defect, wherein the verification data set may include multiple pairs of image groups and multiple pairs of labels corresponding to the pairs of image groups. Then, the electronic device 10 calculates the performance (for example, an indicator associated with a confusion matrix such as accuracy or recall rate) of the third machine learning model 13′ according to a detection result of the verification data set.

In step S608, the electronic device 10 may judge whether the training of the third machine learning model 13′ is completed. If the training is completed, step S610 is executed. If the training is not yet completed, step S609 is executed. For example, the electronic device 10 may judge whether the performance of the third machine learning model 13′ is higher than a requirement. If the performance of the third machine learning model 13′ is higher than the required performance, the electronic device 10 may judge that the training is completed. If the performance of the third machine learning model 13′ is lower than or equal to the required performance, the electronic device 10 may judge that the training is not yet completed.

In step S609, if the training is not yet completed, the electronic device 10 may adjust at least one hyperparameter of the third machine learning model 13′, and retrain the third machine learning model 13′ according to the updated hyperparameter.

In step S610, the electronic device 10 may store the well-trained third machine learning model 13. Specifically, the electronic device 10 packages network framework parameters of the first model 51 (or the second model 52) and the fully connected network 53 and the corresponding neuron weights into one file according to a specific neural network data exchange format to store the well-trained third machine learning model 13.

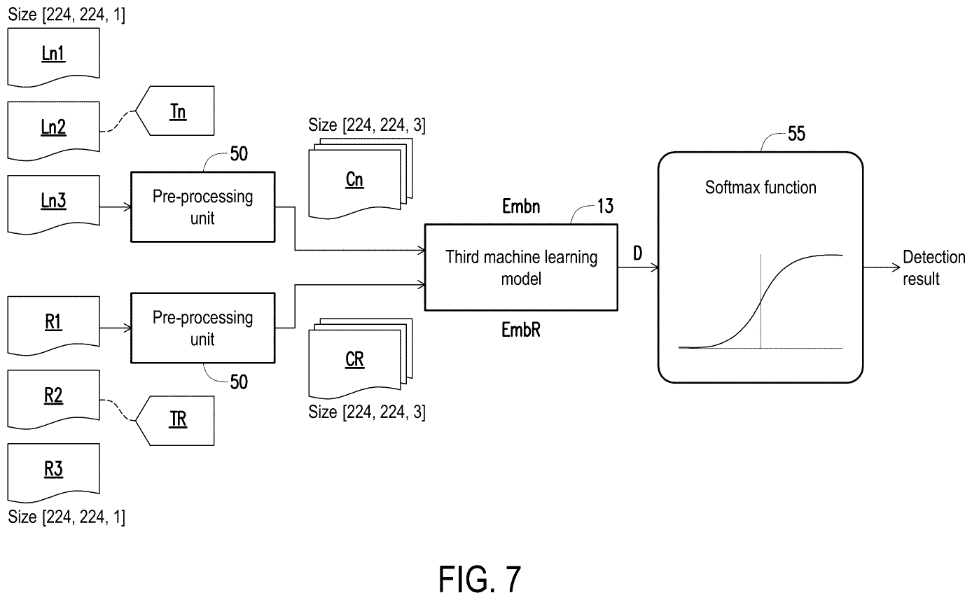

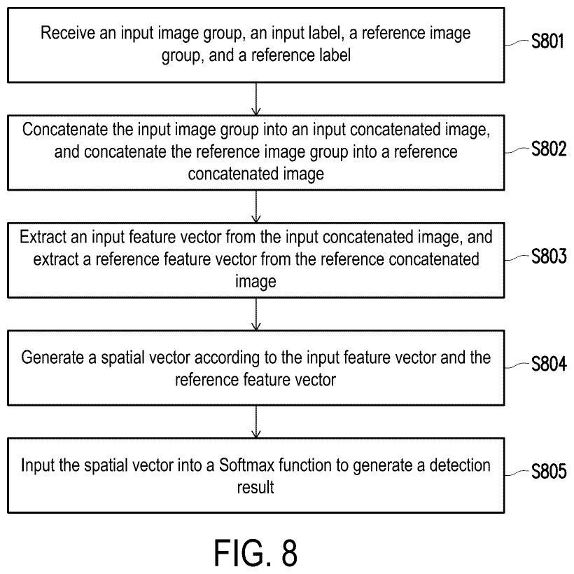

FIG. 7 and FIG. 8 are respectively a schematic diagram and a flowchart of an inference process of the third machine learning model 13 according to an embodiment of the disclosure.

In step S801, the electronic device 10 may receive an input image group, an input label Tn corresponding to the input image group, a reference image group, and a reference label TR corresponding to the reference image group, wherein the input image group may include images Ln1, Ln2, and Ln3 corresponding to different cross-sections of one solder paste, and the reference image group may include images R1, R2, and R3 corresponding to different cross-sections of another solder paste. The input label Tn indicates the bounding box, the solder paste category name, and whether there is a defect corresponding to the input image group. The reference label TR indicates the bounding box, the solder paste category name, and whether there is a defect corresponding to the reference image group.

In step S802, the pre-processing unit 50 of the electronic device 10 may concatenate each image (for example, the image Ln1, Ln2, or Ln3) in the input image group into an input concatenated image Cn, and may concatenate each image (for example, the image R1, R2, or R3) in the reference image group into a reference concatenated image CR.

In an embodiment, the pre-processing unit 50 may enlarge the bounding box on the image group or the concatenated image to update the bounding box. The method for enlarging the bounding box may be similar to the method of step S303, so there will be no reiteration.

In step S803, the electronic device 10 may input the input concatenated image Cn and the reference concatenated image CR into the third machine learning model 13. The first model 51 of the third machine learning model 13 may extract an input feature vector Embn from the input concatenated image Cn. The second model 52 of the third machine learning model 13 may extract a reference feature vector EmbR from the reference concatenated image CR.

In step S804, the fully connected network 53 of the electronic device 10 may generate the spatial vector D according to the input feature vector Embn and the reference feature vector EmbR, wherein the spatial vector D indicates, for example, the Euclidean distance between the input feature vector Embn and the reference feature vector EmbR.

In step S805, the electronic device 10 may input the spatial vector D into a Softmax function 55 to normalize the spatial vector D into a probability distribution summing to 1, wherein the probability distribution indicates the probability of the input image group and the reference image group belonging to the same category. The electronic device 10 may generate a detection result according to the probability distribution. The detection result may indicate whether the solder paste in the input image group has a defect.

Assuming that the reference image group has a defect, if the output of the Softmax function 55 indicates that the probability of the input image group and the reference image group belonging to the same category is greater than a threshold, the detection result generated by the electronic device 10 may indicate that the input image group has a defect. If the output of the Softmax function 55 indicates that the probability of the input image group and the reference image group belonging to the same category is less than or equal to the threshold, the detection result generated by the electronic device 10 may indicate that the input image group does not have a defect.

On the other hand, according to the description of FIG. 1, when the solder paste is detected to have a defect at the first inspection, the AXI machine 14 records “unqualified” and the bounding box corresponding to the defect in the label, and then transmits the image and the label of the solder paste to the electronic device 10. Therefore, during the inference process, all the images of the solder paste in the input image group Tn received by the electronic device 10 are labelled to have defects. Assuming that the reference image group has no defect, if the output of the Softmax function 55 indicates that the probability of the input image group and the reference image group belonging to the same category is greater than the threshold, the detection result generated by the electronic device 10 may indicate that the input image group has no defect. If the output of the Softmax function 55 indicates that the probability of the input image group and the reference image group belonging to the same category is less than or equal to the threshold, the detection result generated by the electronic device 10 may indicate that the input image group has a defect.

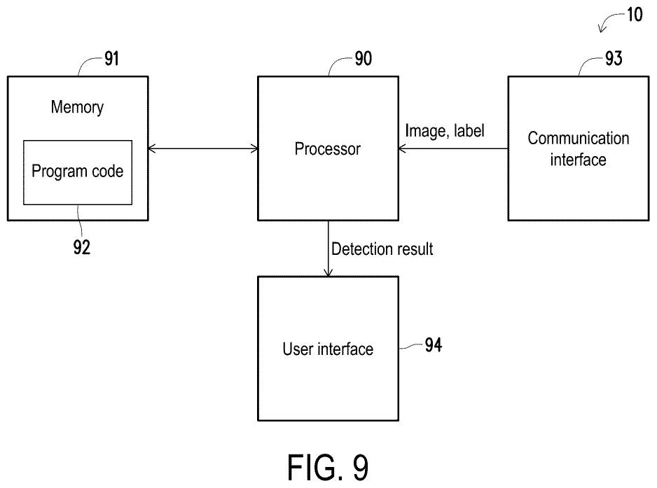

FIG. 9 is a schematic diagram of the electronic device 10 according to an embodiment of the disclosure. The electronic device 10 may include a processor 90, a memory 91, a communication interface 93, and a user interface 94. The processor 90 is electrically connected to the memory 91, the communication interface 93, and the user interface 94, and is configured to access and execute a program code 92 stored in the memory 91 to execute various functions of the electronic device 10. The method 3 for detecting the defect of the solder paste of FIG. 3, the training process of FIG. 6, and the inference process of FIG. 8 may be compiled into the program code 92 to embody modules or application programs such as the first machine learning model 11, the second machine learning model 12, the third machine learning model 13, the pre-processing unit 40, the binarization unit 41, the pre-processing unit 50, the first model 51, the second model 52, or the fully connected network 53.

The processor 90 is, for example, a central processing unit (CPU), other programmable general-purpose or specific-purpose micro control units (MCU), microprocessors, digital signal processors (DSP), programmable controllers, application specific integrated circuits (ASIC), graphics processing units (GPU), image signal processors (ISP), image processing units (IPU), arithmetic logic units (ALU), complex programmable logic devices (CPLD), field programmable gate arrays (FPGA), other similar elements, or a combination of the above elements.

The memory 91 is, for example, any type of fixed or removable random-access memory (RAM), read-only memory (ROM), flash memory, hard disk drive (HDD), solid state drive (SSD), similar elements, or a combination of the above elements, and is configured to store the program code 92 executable by the processor 90.

The communication interface 93 transmits or receives signals wirelessly or by wire. The communication interface 93 may also execute, for example, low noise amplification, impedance matching, mixing, up or down frequency conversion, filtering, amplification, and similar operations. In an embodiment, the processor 90 may receive the image and the corresponding label from an external electronic device (for example, the AXI machine 14) through the communication interface 93.

The user interface 94 is, for example, a human machine interface (HMI) including an input device or an output device. The user interface 94 may output multimedia information such as an image or a sound for user reference. For example, the processor 90 may output the detection result of the image to the user through the user interface 94.

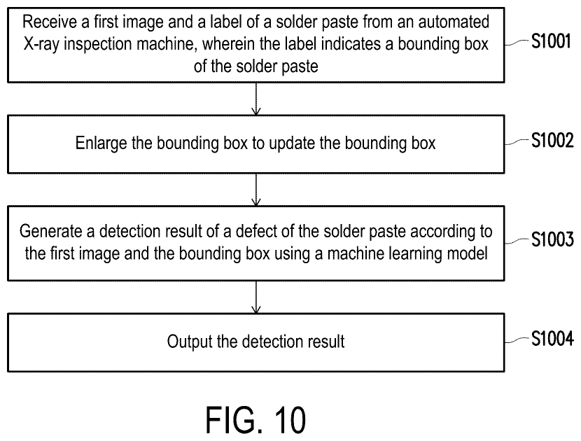

FIG. 10 is a flowchart of a method for detecting a defect of a solder paste according to an embodiment of the disclosure, wherein the method may be implemented by the electronic device 10. In step S1001, a first image and a label of a solder paste are received from an AXI machine, wherein the label indicates a bounding box of the solder paste. In step S1002, the bounding box is enlarged to update the bounding box. In step S1003, a detection result of a defect of the solder paste is generated according to the first image and the bounding box using a machine learning model. In step S1004, the detection result is output.

In summary, the method, the electronic device, and the system for detecting the defect of the solder paste of the disclosure have the following characteristics. (1) The AXI machine and the electronic device are configured to respectively perform defect inspection twice. If the detection result of the second defect inspection is still unqualified, personnel at the manual inspection station will perform the third inspection to prevent unnecessary manual inspection. (2) Multiple machine learning models are configured to respectively perform defect inspection on multiple solder pastes of multiple solder paste groups to solve the issue of the overkill rate of a single machine learning model being too high. (3) Pre-processing such as enlarging the bounding box of the input image may be performed, which may solve the issue of the bounding box generated by the conventional AXI machine being displaced or inaccurate. (4) For a thicker solder paste, such as the ball grid array or the plated through hole, multiple images corresponding to multiple cross-sections are concatenated for image recognition, which may fully inspect the defect of the solder paste. (5) For the thicker solder paste, the weights of the neurons in the two Siamese networks used are exactly the same, which may save the memory spaces occupied by the neural networks.

What is claimed is:

1. A method for detecting a defect of a solder paste, comprising:
receiving a first image and a label of the solder paste from an automated X-ray inspection machine, wherein the label indicates a bounding box of the solder paste;
enlarging the bounding box to update the bounding box;
by a machine learning model, generating a detection result of the defect of the solder paste according to the first image and the bounding box; and
outputting the detection result.

2. The method according to claim 1, wherein the step of by the machine learning model, generating the detection result of the defect of the solder paste according to the first image and the bounding box comprises:
judging that the solder paste corresponds to a first solder paste group according to the label;
in response to the solder paste corresponding to the first solder paste group, executing binarization on a part of the first image in the bounding box to generate a binarized image; and
by the machine learning model, detecting the binarized image to generate the detection result.

3. The method according to claim 2, wherein the step of executing the binarization comprises:
executing the binarization according to Otsu's method.

4. The method according to claim 2, wherein the machine learning model comprises MobileNetV3.

5. The method according to claim 2, wherein the solder paste in the first solder paste group corresponds to universal.

6. The method according to claim 1, wherein the step of by the machine learning model, generating the detection result of the defect of the solder paste according to the first image and the bounding box comprises:
judging that the solder paste corresponds to a second solder paste group according to the label; and
in response to the solder paste corresponding to the second solder paste group, transmitting a part of the first image in the bounding box to the automated X-ray inspection machine, and receiving the detection result corresponding to the part from the automated X-ray inspection machine.

7. The method according to claim 6, wherein the solder paste in the second solder paste group corresponds to one of a quad flat no-lead, a small outline transistor, a gullwing, a ground pad, a resistor, a capacitor, a press-fit, and a chip.

8. The method according to claim 1, wherein the step of by the machine learning model, generating the detection result of the defect of the solder paste according to the first image and the bounding box comprises:
receiving a second image and a third image of the solder paste from the automated X-ray detector, wherein the first image, the second image, and the third image respectively correspond to different cross-sections of the solder paste;
judging that the solder paste corresponds to a third solder paste group according to the label;
in response to the solder paste corresponding to the third solder paste group, concatenating the first image, the second image, and the third image to generate a concatenated image; and
generating the detection result according to the concatenated image.

9. The method according to claim 8, wherein the machine learning model comprises a Siamese network model, wherein before the step of generating the detection result according to the concatenated image, the method further comprises performing a training process, comprising:
extracting a first feature vector from a first concatenated image by a first model in the Siamese network model;
extracting a second feature vector from a second concatenated image by a second model in the Siamese network model; and
generating a spatial vector according to the first feature vector and the second feature vector by a fully connected network.

10. The method according to claim 9, wherein the training process further comprises:
calculating a probability vector of a loss function according to the spatial vector; and
updating the first model and the second model according to the probability vector.

11. The method according to claim 10, wherein the step of updating the first model and the second model according to the probability vector comprises:
adjusting a first weight of the first model and a second weight of the second model based on weight sharing.

12. The method according to claim 10, wherein the loss function comprises a contrastive loss function.

13. The method according to claim 8, wherein the step of generating the detection result according to the concatenated image comprises:
executing a Softmax function on an output of the machine learning model to generate the detection result.

14. The method according to claim 8, wherein the solder paste in the third solder paste group corresponds to one of a ball grid array and a plated through hole.

15. An electronic device for detecting a defect of a solder paste, comprising:
a communication interface, configured to receive at least one image and a label of the solder paste;
a processor, electrically connected to the communication interface; and
a memory, electrically connected to the processor and configured to store a program code, wherein the program code instructs the processor to execute the method for detecting the defect of the solder paste according to claim 1 according to the at least one image and the label.

16. A system for detecting a defect of a solder paste, comprising:
an automated X-ray inspection machine, configured to capture at least one image of the solder paste of a circuit board and generate a label of the at least one image;
the electronic device according to claim 15, wherein the automated X-ray inspection machine is configured to generate a detection result of the defect of the solder paste according to the at least one image and the label;
an electronic inspection station, connected to the electronic device and configured to receive the circuit board when the detection result indicates “qualified”; and
a manual inspection station, connected to the electronic device and configured to receive the circuit board when the detection result indicates “unqualified”.
